(19) United States
(12) Patent Application Publication
(10) Pub. No.: US 2020/0289271 A1
(43) Pub. Date: Sep. 17, 2020

(54) MODULAR MANDIBULAR PROSTHESIS

(71) Applicant: Omega Innovations, Inc., San Diego, CA (US)

(72) Inventor: Stacey Michelle Nedrud, Jacksonville, FL (US)

(21) Appl. No.: 16/817,345

(22) Filed: Mar. 12, 2020

Related U.S. Application Data
(60) Provisional application No. 62/816,955, filed on Mar. 12, 2019.

(51) Int. Cl. A61F 2/28

(52) U.S. Cl. CPC A61F 2/2803; A61F 2002/30263

(57) ABSTRACT

Devices and methods for modular mandibular prostheses. The prosthesis includes a plurality of connector links that can be connected together in a desired size and orientation. For example, a ball and socket may provide the ability to configure to most 3D configuration. Each link has a body with engagement structures on first and/or second ends of the body. The engagement structure on the first end of a first link may be received by the engagement structure on the second end of a second link. The connected links can be in a locked and unlocked state. An expansion or compression member engages the connected link to cause the prosthesis to be in the locked state. An end attachment may be used on ends of outermost connected links to attach the prosthesis to a patient's mandible.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. For example, this application claims the benefit of priority of U.S. Provisional Application No. 62/816,955, filed Mar. 12, 2019, and titled “Nedrud Modular Prosthesis,” the entirety of which is incorporated by reference herein for all purposes and forms a part of this specification.

### BACKGROUND

### Field

The technology relates to prosthetics, in particular to mandibular prosthetics.

### Description of the Related Art

A mandibulectomy or segmental mandibulectomy is a medical procedure involving removal of part of the jaw bone, also known as the mandible. This may be done when, for example, benign or malignant pathology, trauma, among others is involved with or close to involving the mandible. The defective portion is then reconstructed and replaced with a prosthetic, autograft, allograft, synthetic graft mandible or portion thereof. Existing solutions to replacing the defective portion include replacement with metal plates and/or bone from another area of the body. For example the surgeon can use bone from the patient with or without its own blood supply, or simply bridge the bony gap with a titanium plate and screws. However, there are drawbacks to using bone, including morbidity of harvesting tissue from another site in the body, the time that it takes to perform the surgery, and its associated costs. Further, using a titanium plate and screws alone, without further reconstruction, compromises establishment of a desired cosmetic form, which is a major tenet of reconstruction.

Another approach to reconstructing a mandibulectomy includes custom made Virtual Surgically Planned (VSP) titanium mandibular reconstruction, with or without grafting. However, great time and expense is required to image the mandible and 3D print the prosthesis. The major drawback of this alternative is the time and expense required.

There is therefore a need for improvements to existing solutions to segmental mandibulectomies.

### SUMMARY

The embodiments disclosed herein each have several aspects, no single one of which is solely responsible for the disclosure's desirable attributes. Without limiting the scope of this disclosure, its more prominent features will now be briefly discussed. After considering this discussion, and particularly after reading the section entitled “Detailed Description of the Preferred Embodiments,” one will understand how the features of the embodiments described herein provide advantages over existing approaches to mandibular prostheses.

The devices and methods described herein may be used in patients with segmental mandibular defects (e.g. missing segments of the lower jaw). The development includes a modular mandibular (lower jaw) prosthetic that can be easily adapted to any shape and location of the mandible. A prosthesis or portion thereof may be custom built using modular links attached together to a desired length and configuration. A plurality of connector links may be connected together in a desired size and orientation. Each link has a body with engagement structures, which may be on one or both of first and second ends of the body. The engagement structure on the first end of a first link is received by the engagement structure on the second end of a second link. The connected links can be in a locked and unlocked state. An expansion or compression member engages the connected link to cause the prosthesis to be in the locked state. An end attachment may be used on ends of outermost connected links to attach the prosthesis to a patient's mandible.

The prosthesis can be used as either a temporary place holder prior to definitive reconstruction, or as a permanent solution with a future possibility of definitive reconstruction with dental implants. In a select group of patients needing palliative resection, it can serve as the final reconstruction, thus relieving the patient of the morbidity of reconstruction with autogenous methods (i.e., using the patient's own tissues). The “building block” approach to mandibular prosthetics described herein may also be used for other prosthetics in other parts of the body.

In one aspect, a modular mandibular prosthetic for replacing at least a portion of a patient's mandible is described. The prosthetic comprises at least a first connector link and a second connector link, and an expansion or compression member. Both of the first and second connector links form at least a portion of the modular mandibular prosthetic. The modular mandibular prosthetic has an unlocked state and a locked state. Each of the first and second connector links has a body, a first end and a second end. The first end forms a first engagement structure and the second end forms a second engagement structure. The first engagement structure of the first connector link is sized and shaped to be received by the second engagement structure of the second connector link. The expansion or compression member is configured to engage with at least one of the first connector link and the second connector link when the first engagement structure of the first connector link is received by the second engagement structure of the second connector link to move the modular mandibular prosthetic from the unlocked state to the locked state.

Various embodiments of the various aspects are described. A level of resistance to moving the first connector link relative to the second connector link may increase when the modular mandibular prosthetic is moved from the unlocked state to the locked state. The body of each of the first connector link and the second connector link may have a general cuboidal shape. The body of each of the first connector link and the second connector link may have a unitary structure. The body of each of the first connector link and the second connector link may have a monolithic structure. The first engagement structure of the first connector link may have a convex spherical shape and the second engagement structure of the second connector link may have a concave spherical shape. The first engagement structure of the first connector link may have a ball shape and the second engagement structure of the second connector link may have a socket shape.

The body of the first connector link may have a receptacle disposed between the first end and the second end, the receptacle being sized and shaped to receive a dental implant. The receptacle may be disposed between the first engagement structure and the second engagement structure. The modular mandibular prosthetic may further comprise at least one end attachment, the at least one end attachment being configured to connect an end of the portion of the modular mandibular prosthetic to a portion of the patient's mandible. The at least one end attachment may have a generally planar shape. The second engagement structure of the first connector link may be sized and shaped to receive the first engagement structure of the second connector link.

The expansion or compression member may be a screw. The expansion or compression member may engage with the first connector link by rotating the expansion or compression member relative to the first connector link. The expansion or compression member may engage with the body of the first connector link by penetrating the body. The expansion or compression member may penetrate the first engagement member of the first connector link at a location. The location may be between the body of the first connector link and the body of the second connector link. The expansion or compression member may engage with the second connector link by rotating the expansion or compression member relative to the second connector link.

The first connector link may be movable with respect to the second connector link when the modular mandibular prosthetic is in the unlocked state. The movement of the first connector link may be a swivel. The swivel may be in three dimensions. The first connector link may be fixed with respect to the second connector link when the portion of the modular mandibular prosthetic is in the locked state.

The second engagement structure of the second connector link may be sized and shaped to receive the first engagement structure of the first connector link in at least a first position and a second position so that an overall length of the first connector link and the second connector link when the modular mandibular prosthetic is in the first position is greater than an overall length of the first connector link and the second connector link when the modular mandibular prosthetic is in the second position. Moving between the first position and the second position may be achieved by rotating the first connector link relative to the second connector link. Moving between the first position and the second position may be achieved by axially moving the first connector link relative to the second connector link.

The expansion or compression member may be releasable. The portion of the modular mandibular prosthetic may maintain an assembled state when the modular mandibular prosthetic is in the unlocked state and the expansion or compression member is disengaged from both of the first connector link and the second connector link. The patient's mandible may have a lingual surface and a facial surface, and the first engagement structure and the second engagement structure of each of the first connector link and the second connector link may lie in a plane between the lingual surface and the facial surface.

In another aspect, a modular mandibular prosthetic for replacing at least a portion of a patient's mandible is described. The patient's mandible has a lingual surface and a facial surface. The prosthetic comprises at least a first connector link and a second connector link. Both of the first and second connector links form at least a portion of the modular mandibular prosthetic. The modular mandibular prosthetic has an unlocked state and a locked state. Each of the first and second connector links has a body, a first end and a second end. The first end forms a first engagement structure and the second end forming a second engagement structure. The first engagement structure of the first connector link is configured to be received by the second engagement structure of the second connector link. The first engagement structure and the second engagement structure lie in a plane between the lingual surface and the facial surface. The modular mandibular prosthetic further comprises an expansion or compression member configured to engage with at least one of the first connector link and the second connector link when the first engagement structure of the first connector link is received by the second engagement structure of the second connector link.

In another aspect, a modular mandibular prosthetic for replacing at least a portion of a patient's mandible is described. The modular mandibular prosthetic has an unlocked state and a locked state. The modular mandibular prosthetic comprises a first connector link that has a body, a first end and a second end. The first end forms a first engagement structure and the second end forms a second engagement structure. The first connector link forms at least a first portion of the modular mandibular prosthetic. The modular mandibular prosthetic further comprises a second connector link that has a body, a first end and a second end. The first end forms a first engagement structure and the second end forms a second engagement structure. The second connector link forms at least a second portion of the modular mandibular prosthetic. The first engagement structure of the first connector link is sized and shaped to be received by the second engagement structure of the second connector link when the modular mandibular prosthetic is in both the unlocked state and the locked state.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

FIG. 1 is a perspective view of an embodiment of a modular mandibular prosthetic having connecting links with spherical engagement structures and shown connecting two portions of a mandible.

FIG. 2 is a perspective view of another embodiment of a modular mandibular prosthetic having connecting links with spherical engagement structures and shown connecting a mandible with a temporomandibular joint.

FIGS. 3A and 3B are perspective views of various configurations achievable by various embodiments of a modular mandibular prosthetic.

FIGS. 3C and 3D are photographs of various prototype embodiments of a modular mandibular prosthetic.

FIGS. 4 and 5 are perspective views of embodiments of a modular mandibular prosthetic having spherical and mushroom shaped engagement structures respectively and shown disconnected.

FIGS. 6A and 6B are various views of embodiments of connecting links that may be used with the various modular mandibular prosthetics described herein.

FIGS. 7A-7C are perspective views of another embodiment of a modular mandibular prosthetic having swivel engagement structures and shown in various configurations.

FIG. 8 is a perspective view of another embodiment of a modular mandible prosthetic having swivel engagement structures and shown disconnected.

FIG. 9A is a perspective view of an embodiment of a connecting link having a receptacle configured to receive a dental implant. In certain embodiments, the connecting link already incorporates the dental implant. The dental implant is also referred to as a screw or cylinder. A dental crown can be subsequently placed on the dental implant or screw. The connecting link may be used with the various modular mandibular prosthetics described herein.

FIG. 9B is perspective view of the connecting link of FIG. 9A having the dental implant received into the receptacle with the dental crown secured thereto.

FIG. 10 is a perspective view of an embodiment of a connecting link having a spherical engagement structure with various locations configured to receive an expansion or compression member and that may be used with the various modular mandibular prosthetics described herein.

FIG. 11 is a perspective view of an embodiment of a modular mandibular prosthetic with a connecting link having a mushroom shaped engagement structure with various locations configured to receive an expansion or compression member and that may be used with the various modular mandibular prosthetics described herein.

FIGS. 12A and 12B are sequential side views of a spherical engagement structure before and after tightening of an expansion or compression member into the engagement structure.

FIGS. 13A and 13B are cross-section views of two connecting links having engagement structures which allow axial fine adjustments of length, and shown in first and second axial positions.

FIGS. 14A and 14B are cross-section views of two connecting links having engagement structures with first and second positioning portions for coarse adjustments and shown in first and second axial positions.

FIGS. 15A and 15B are various views of an embodiment of a connecting link having various trabeculated tissue surfaces to encourage mandible tissue ingrowth, or for placement of graft material.

FIG. 16 is a perspective view of an embodiment of a body having a hollow structure with a system of trusses for stability and to decrease the weight, and encourage mandibular tissue ingrowth or graft material. The body may be used with the various modular mandibular prosthetics described herein.

FIGS. 17A and 17B are sequential side views of an embodiment of a tool that may be used to connect the links together for the various modular mandibular prosthetics described herein.

FIG. 18 is a perspective view of another embodiment of a modular mandibular prosthetic having an integral mandible attachment at ends thereof and shown prior to connecting the links together.

### DETAILED DESCRIPTION OF THE PREFERRED EMBODIMENTS

The following detailed description is directed to certain specific embodiments of the development. Reference in this specification to “one embodiment,” “an. embodiment,” or “in some embodiments” means that a particular feature, structure, or characteristic described in connection with the embodiment is included in at least one embodiment of the invention. The appearances of the phrases “one embodiment,” “an embodiment,” or “in some embodiments” in various places in the specification are not necessarily all referring to the same embodiment of the prosthetic, nor are separate or alternative embodiments necessarily mutually exclusive of other embodiments. Moreover, various features are described which may be exhibited by some embodiments and not by others. Similarly, various requirements are described which may be requirements for some embodiments but may not be requirements for other embodiments.

Various embodiments will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments of the development. Furthermore, embodiments of the development may include several novel features, no single one of which is solely responsible for its desirable attributes or which is essential to practicing the invention described herein.

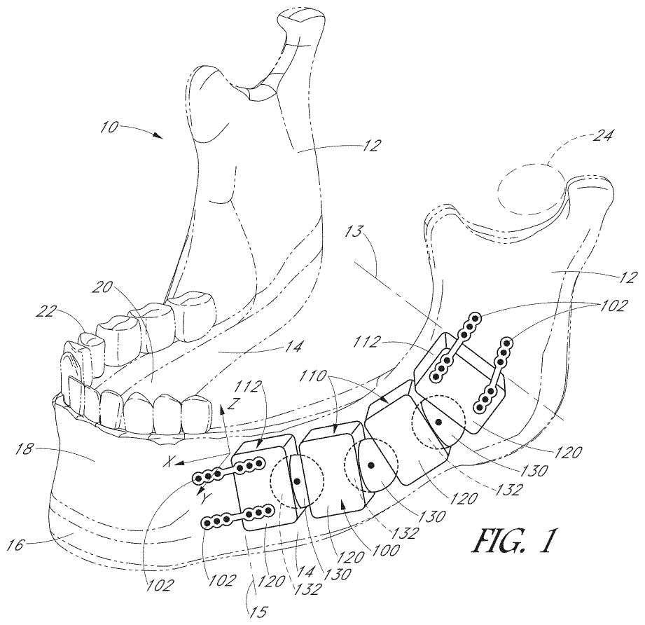

FIG. 1 is a perspective view of an embodiment of a modular mandibular prosthetic 100. In certain embodiments, the prosthetic 100 includes connecting links 110, 112. In certain embodiments, the connecting links 110, 112 include spherical engagement structures 130. The prosthetic 100 is shown connecting two portions of a mandible 10. Any features described with respect to an embodiment of the prosthetic may apply to any other embodiment. Thus, any features of the prosthetic 100 may apply to other embodiments described herein, and vice versa.

The prosthetic 100 may be used for a mandibulectomy or segmental mandibulectomy (used interchangeably herein) A mandibulectomy is the removal of all or part of the mandible 10 or jawbone. The mandibulectomy may be used in various settings, including infectious etiologies (e.g., osteomyelitis), osteoradionecrosis, or a benign (e.g., ameloblastoma) or malignant neoplastic process (e.g., invasive squamous cell carcinoma) that involves the jaw. A mandibulectomy may be used for severe oral and maxillofacial trauma if a section of the mandible 10 is not salvageable.

As shown in FIG. 1, the prosthetic 100 is shown implanted with remaining portions of the mandible 10. The mandible 10 forms the lower jaw and holds the lower teeth 22 in place. The mandible sits beneath the maxilla. The mandible 10 is a single bone in articulation with the skull by bilateral temporomandibular joints 24.

The mandible 10 includes bilateral condylar heads (joints) and necks, bilateral rami 12 connected to bilateral mandibular bodies 14 and that are located on either side of an anterior symphyseal region that includes the chin 16. The mandible 10 includes an outer (lateral) or facial surface 18 and an opposite inner (medial) or lingual surface 20. The mandible 10 has a largely consistent curved shape in multiple planes. The temporomandibular joint 24 is a hinged portion that articulates the mandible 10 to the temporal bones of the skull, which are in front of each ear.

The prosthetic 100 is shown connecting a forward portion of the body 14 (or an outer portion of the chin 16) to the adjacent ramus 12. The prosthetic 100 may be implanted into existing tissue of the patient that surrounds the prosthetic 100. In some embodiments, the entire bone and tissue of the defective portion of the mandible 10 may be removed completely and replaced by the prosthetic 100, and either biocompatible tissue replacement and/or natural tissue may be placed or caused to grow around the prosthetic 100. The mandible 10 may have a section between references 13 and 15 removed or partially removed, and the prosthetic 100 may be implanted between the references 13 and 15. The prosthetic 100 may extend from the reference 13 to the reference 15. The prosthetic 100 may extend along this direction and have any desirable arc length.

The various embodiments of prosthetics described herein, such as the prosthetic 100, provide various uniquely desirable features for mandibulectomies and other suitable procedures. For example, in certain embodiments, the prosthetic 100 provides a “buildable” prosthetic to fit any mandibular defect and that may be less expensive than existing prosthetics. In certain embodiments, the prosthetic 100 can be sized to an average mandible so that the prosthetic 100 has wide patient applicability. In certain embodiments, the prosthetic 100 better re-establishes mandibular width and height, as well as the esthetic curvature of the mandible 10. In certain embodiments, the prosthetic 100 is better able to withstand forces of mastication without fracture, compared to a titanium plate alone. In certain embodiments, the prosthetic 100 has a finite number of “stock” pieces needed to recreate the missing portion of the mandible 10. In certain embodiments, the prosthetic 100 has the possibility for various iterations and applications, such as the possibility for integrating implants. In certain embodiments, the prosthetic 100 may simplify the reconstructive algorithm for mandibular defects. In certain embodiments, the prosthetic 100 may be used for palliative and staged reconstruction where the morbidity of conventional methods are outweighed by their inherent benefits. Use of the prosthetic 100 may supplant the need for autogenous reconstruction.

The prosthetic 100 may find applicability in the reconstruction of defects in other regions of the body, since the concept is based on a simple, low-cost module that offers innumerable options in reconstructing varied bony defects while minimizing morbidity to patients.

The structure of the prosthetic 100 may include two or more inter-articulating base pieces or connecting inner links 110. The prosthetic may include one or more end links 112. As shown, the prosthetic 100 includes three inner links HO and two end links 112. There may be one, two, three, four, five, six, seven, eight, nine, ten, or more inner links 110. There may be zero, one, or two end links 112. Each link 110, 112 includes a body 120. One or more of the links 110, 112 includes an engagement structure 130. The inner links 110 may be connected using the engagement structures 130 to a form a desired length, and the end links 112 may be connected and located on the two ends of a formed string of connected inner links 110. The width and height of the links 110, 112 may be based on an anatomical average of mandibular widths and heights, which may eliminate dead-space.

In certain embodiments, the body 120 has a generally cubed or cuboidal shape as shown. In other embodiments, the body 120 has a cylindrical shape. Of course the shape of the body 120 is not limited and can have any other desirable shape. The corners and edges of the body 120 can be chamfered, shaped, and/or rounded to prevent clinical issues such as, for example, damage to overlying tissue (dehiscence).

The body 120 may be formed of a biocompatible material, or may be used in conjunction with novel tissue engineering. The body 120 may be a unitary structure. The body 120 may be a monolithic structure. For example, the body 120 may be formed from a single piece of material or additively manufactured. In certain embodiments, the body 120 has six faces. In certain embodiments, the body 120 may have a square or rectangular face or faces.

For the sake of description, a local reference XYZ axis system is shown in FIG. 1 relative to the forward/left-most end link 112. Similar reference systems may be applied to the other links 110, 112. The axis system includes an X axis extending along a first length of the body 120, a Y axis extending outwardly away from the mandible 10, and a Z axis that is perpendicular to the X and Y axes. In certain embodiments, the body 120 has a cuboidal shape that is longer in the Z-direction than the X-direction, which is longer than the Y-direction. Any other relationship between the lengths of the sides of the body 120 may be implemented. For example, the ascending or descending order of the lengths of the sides of the body 120 may be X, Y, Z, or X, Z, Y, or Y, Z, X, or Y, X, Z, or Z, Y, X, or Z, X, Y. Two or more of any of these lengths may be the same or substantially the same length. In some embodiments, all three of the lengths of the sides of the body 120 may be equal or substantially equal. As mentioned above, the bodies 120 may have cuboidal shapes with chamfered edges, other suitable shapes, or combinations thereof as further described herein.

The design may use various types of engagement structures 130, such as a spherical connector as shown, to connect the links 110, 112 together. The engagement structure 130 has a convex spherical shape. The engagement structure 130 may therefore bulge or otherwise extend outward away from the body 120 and have a spherical or otherwise rounded outer surface.

The engagement structure 130 may be attached at a first end of the body 120 and connect with a receiving portion 132 at a second end of an adjacent body 120. The engagement structure 130 may be a first engagement structure and the receiving portion 132 may be a second engagement structure that engage with each other to connect two adjacent links 110, 112. The receiving portion 132 may have a corresponding concave shape. The receiving portion 132 may therefore extend inward into the body 120 and form a spherical or otherwise rounded inward surface. Each of the inner links 110 may include the engagement structure 130 on the first end of the body 120 and the corresponding receiving portion 132 on a second opposite end of the body 120. When implanted, the engagement structure 130 and the receiving portion 132 of each of the links 110, 112 may be located between the lingual surface 20 and the facial surface 18. The engagement structure 130 and the receiving portion 132 of each of the links 110, 112 may lie in a plane located between the lingual surface 20 and the facial surface 18.

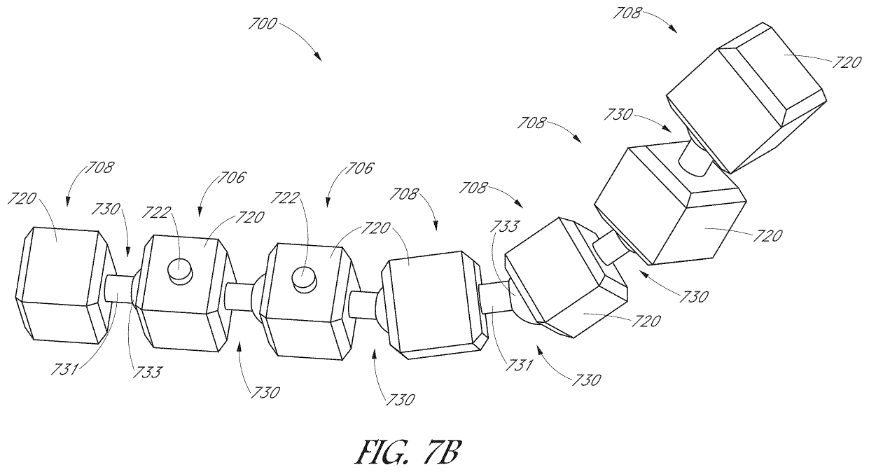

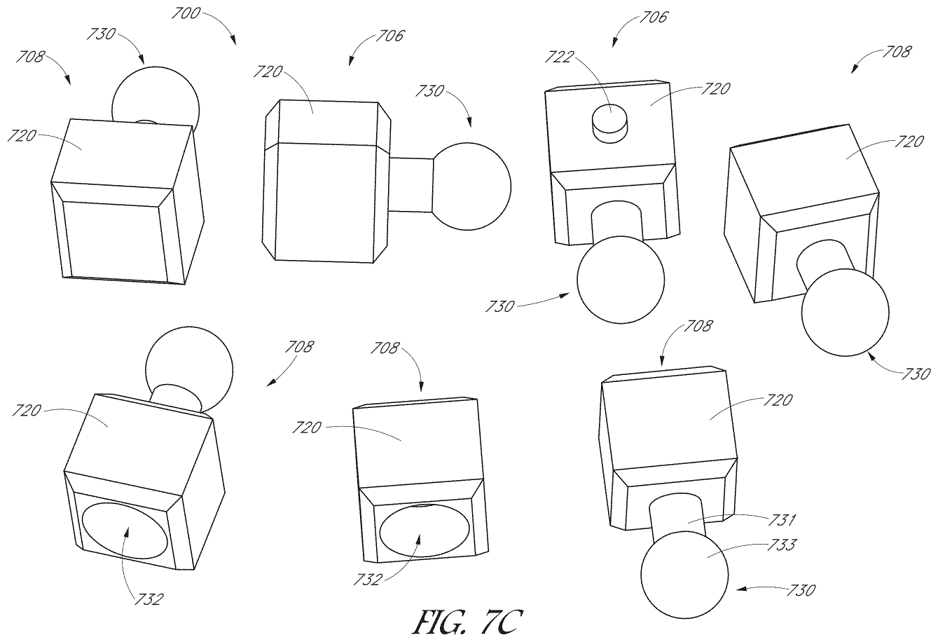

The receiving portion 132 receives and attaches with an adjacent engagement structure 130. The engagement structure 130 may be secured with the receiving portion 132 using a variety of different expansion or compression members, as further described herein. This and the other engagement designs described herein allow for a three-dimensional rotational movement of the links 110, 112, allowing for the reconstruction of the missing portion of the mandible 10 while recreating its natural curvature, with subsequent locking and rigidizing of the prosthetic 100 in a desired and final configuration. The engagement structure 130 and receiving portion 132 may be implemented as a configurable and securable “ball and socket” connection mechanism. In certain embodiments, a ball and socket mechanism can allow for free movement to form any curvature. In some embodiments, the engagement structure 130 may have a ball shape and the receiving portion 132 may have a socket shape, as further described herein, for example with respect to FIGS. 7-10.

In certain embodiments, the end links 112 may include the receiving portion 132 on a first end of the body 120. The end links 112 may not have the engagement structure 130 on the opposite second end of the body 120. In certain embodiments, the second end of the body 120 may be configured to be attached to one or more end attachments 102, such as the elongated, planar attachment members as shown, to attach to the mandible 10.

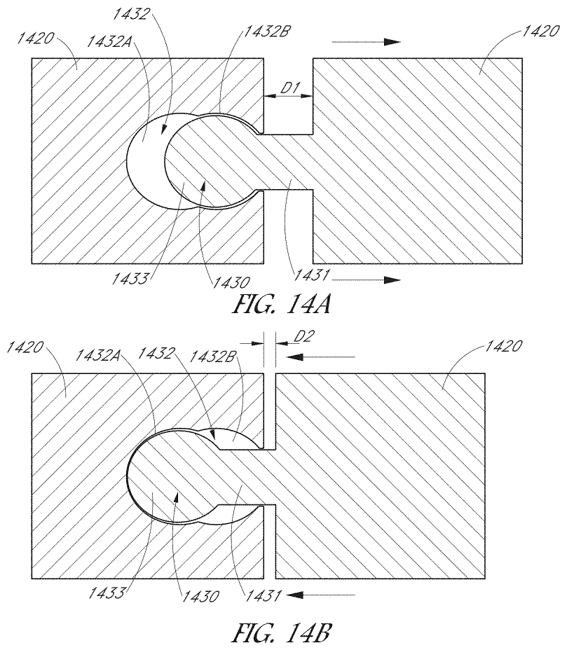

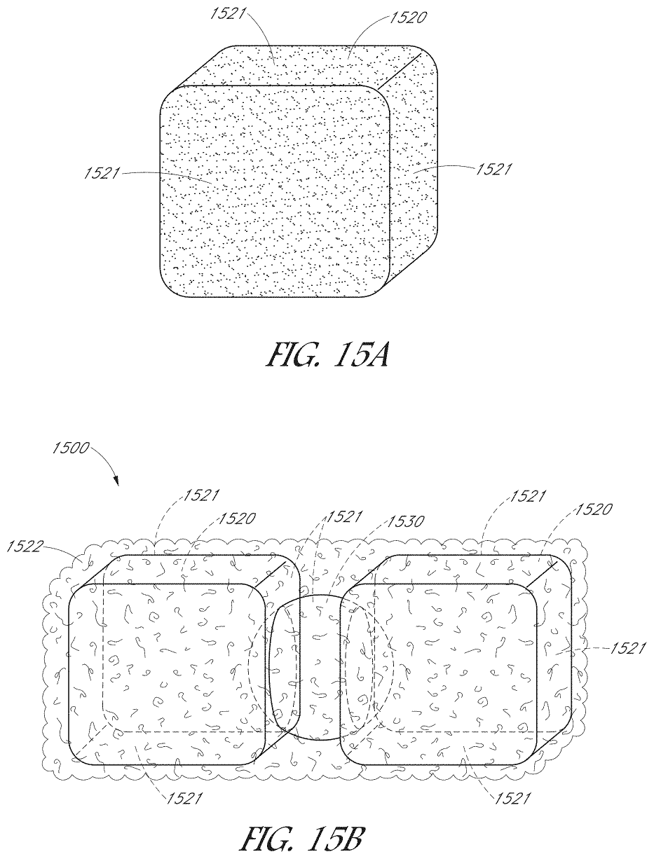

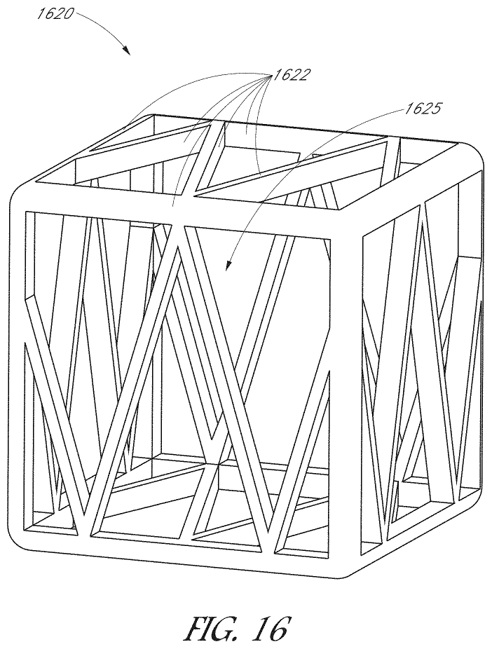

In certain embodiments, the sizing and use of the prosthetic 100 may be based on measurement of the mandibular defect. In certain embodiments, the surgeon may then assemble the needed links 110, 112 to ill the defect while re-establishing the mandibular form, rotating the pieces at the ball-and-socket interface. In certain embodiments, once satisfied with the reconstruction, the surgeon then locks the links 110, 112 in place to render the reconstruction rigid. In certain embodiments, the links 110, 112 may be locked by using an expansion system where a pre-placed expansion or compression member, such as a screw (see FIGS. 14-15B), at the center of the engagement structure 130 is tightened to cause microexpansion of the body 120 of the links 110, 112, and thus locking the links 110, 112 along the friction interface. In some embodiments, a compression system may be used where expansion or compression members themselves are pre-placed in the bodies 120, such as cuboidal prisms, and when tightened will compress the engagement structure 130, such as a sphere, in place.

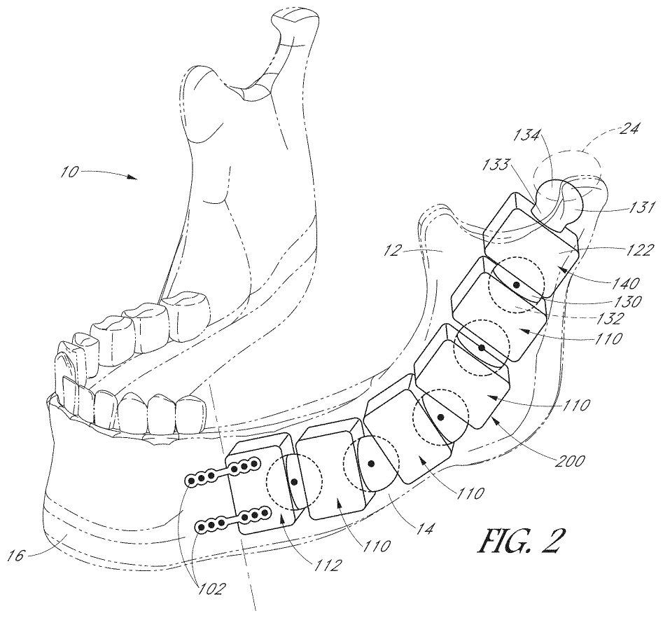

FIG. 2 is a perspective view of another embodiment of a modular mandibular prosthetic 200. The prosthetic 200 may have the same or similar features as the prosthetic 100, and vice versa. In certain embodiments, the prosthetic 200 includes four of the connecting inner links 110. In certain embodiments, the prosthetic 200 at a first end thereof includes one end link 112 attached to the body 14 (or chin portion 16 or anywhere in the mandible) by the end attachments 102. In certain embodiments, the prosthetic 200 at a second end thereof includes another embodiment of an end link 140. The end link 140 includes a body 122. The body 122 includes the engagement structure 130. The engagement structure 130 is located on a first end of the body 122. The engagement structure 130 engages with the receiving portion 132 of the adjacent inner link 110. In certain embodiments, the adjacent inner link 110 receives an expansion or compression member as described to lock the inner link 110 in place with the end link 140. In certain embodiments, the end link 140 may also receive an expansion or compression member. In some embodiments, the end link 140 may include the receiving portion 132, and the adjacent inner link 110 may include the engagement structure 130.

In certain embodiments, the end link 140 includes another embodiment of an attachment 131. In certain embodiments, the attachment 131 has a bulb shape. For example, the attachment 131 can include a narrow necked region 133 extending from the body 120 to an outer and wider spherical end portion 134. The attachment 131 may have a variety of other suitable shapes and configurations. The attachment 131 is located on an end of the body 122 of the end link 140 that is opposite the adjacent inner link 110. In certain embodiments, the attachment 131 extends outwardly from the end link 140. In certain embodiments, the attachment 131 is configured to become a replacement of the condylar head portion of the temporomandibular joint (TMJ) 24. The TMJ 24 is shown in schematic form for clarity. In certain embodiments, the attachment 131 may instead attach to the mandible 10. In certain embodiments, the attachment 131 may attach to the ramus 12. In some embodiments, the prosthetic 200 may include the end link 112 in place of the end link 140.

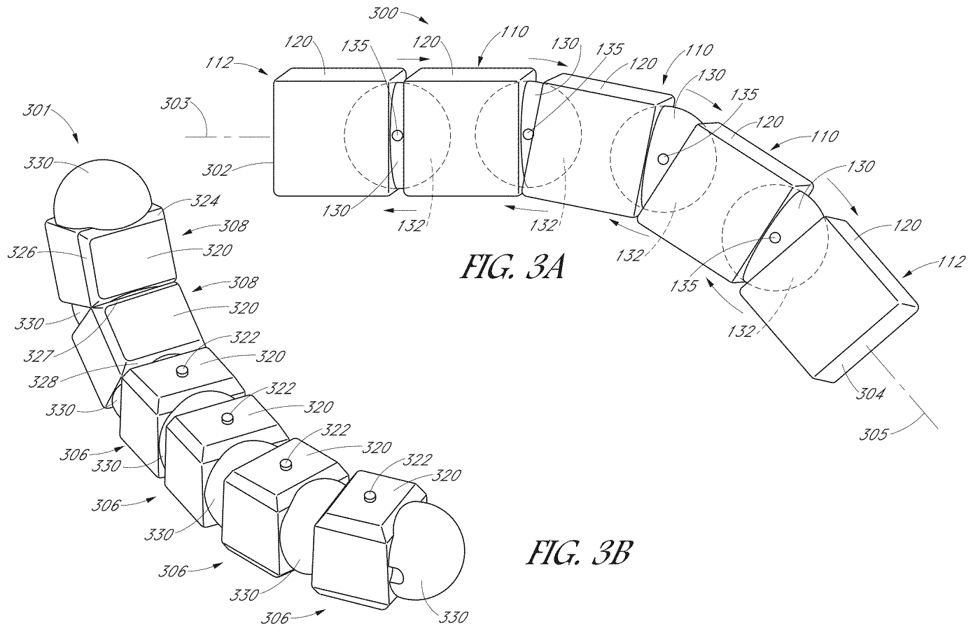

FIGS. 3A and 3B are perspective views of other embodiments of a modular mandibular prosthetic 300 and 301, respectively. The prosthetics 300, 301 may have the same or similar features as any other prosthetic described herein, such as the prosthetics 100 and/or 200, and vice versa.

As shown in FIG. 3A, the prosthetic 300 includes three of the inner links 110 and two end links 112. The bodies 120 of the links 110, 112 are connected in series and have the engagement structures 130 secured with corresponding receiving portions 132 of the adjacent links 110 or 112. The prosthetic 300 has been configured to have a bent shape as shown. Prior to locking the prosthetic 300, the links 110, 112 may extend from a first end 302 to a second end 304. Central axes 303 and 305 defined respectively by the end links 112 at the ends 302 and 304 may be angled with respect to each other.

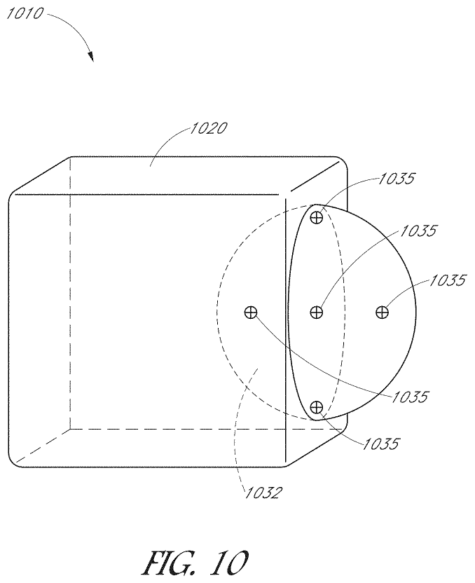

In certain embodiments, the engagement structures 130 each further include an opening 135. In certain embodiments, the opening 135 is configured to receive an expansion or compression member therein to lock the prosthetic 300 in a desired configuration. In certain embodiments, the opening 135 may be threaded or smooth. In certain embodiments, the opening 135 may receive a fastener such as a screw, or the screw may be pre-placed at a depth just short of maximum depth, so that with small additional turns of the screw to further seat the screw, will provide the microexpansion necessary to lock the embodiment in place. In certain embodiments, the opening 135 may expand slightly after receiving the expansion or compression member to cause the prosthetic 300 to assume a locked configuration. Further details of example expansion or compression members and corresponding openings in the engagement structures for the expansion or compression members are further described herein, for example with respect to FIGS. 11-15B.

As shown in FIG. 3B, the prosthetic 301 includes a link 306 having a body 320 and an engagement structure 330. There may be four links 306 as shown, or greater or fewer than four. In certain embodiments, the links 306 each include a projection 322 extending upwardly from a corresponding body 320. The projections 322 may be configured to attach with a prosthetic tooth.

In certain embodiments, the prosthetic 301 includes a link 308 having the body 320 and the engagement structure 330. There may be two links 308 as shown, or greater or fewer than two. The links 308 may be attached to the end of the connected links 306. Any combination, number and order of the links 306 and 308 may be implemented.

The engagement structures 330 may secure with adjacent engagement structures, such as receiving portions, as described. In certain embodiments, the engagement structures 330 may be formed of compliant material. The material may be curable to transition from compliant to rigid after sizing or after implantation. The engagement structures 330 may extend from both ends of the prosthetic 310 as shown, or from only one or none of the ends.

The bodies 320 include chamfered edges 324, 326, 327, 328 on a top end of the bodies 320. The lower opposite end of the bodies 320 also include the chamfered edges. All four sides of a face of the body 320 may include the chamfered edges 324, 326, 327, 328 as shown. In some embodiments, one, two, three, or none of the edges of a side of the body 320 may include the chamfered edges 324, 326, 327, 328. The corners and edges of the body 320 can be chamfered, shaped, and/or rounded to prevent clinical issues such as, for example, damage to overlying tissue (dehiscence).

FIGS. 3C and 3D are photographs of other embodiments of a modular mandibular prosthetic 307 and 309, respectively. The prosthetics 307, 309 may have the same or similar features as any other prosthetic described herein, such as the prosthetics 100, 200, 300, and/or 301, and vice versa. In certain embodiments, the prosthetic 307 includes the links 306 and 308 connected using engagement structures 130. In certain embodiments, the prosthetic 309 includes the links 110 and 112 connected using engagement structures 130. The bodies of the links 110, 112 include chamfered edges, as described with respect to FIG. 3B.

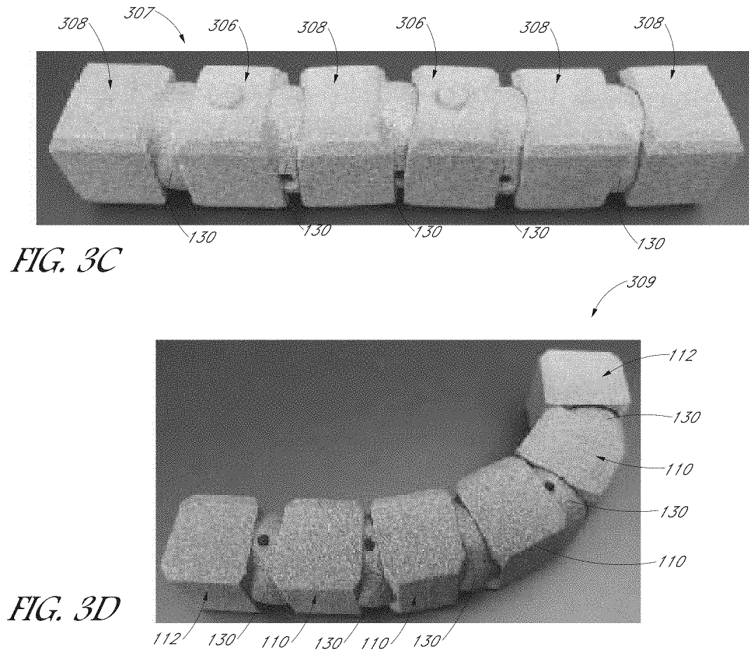

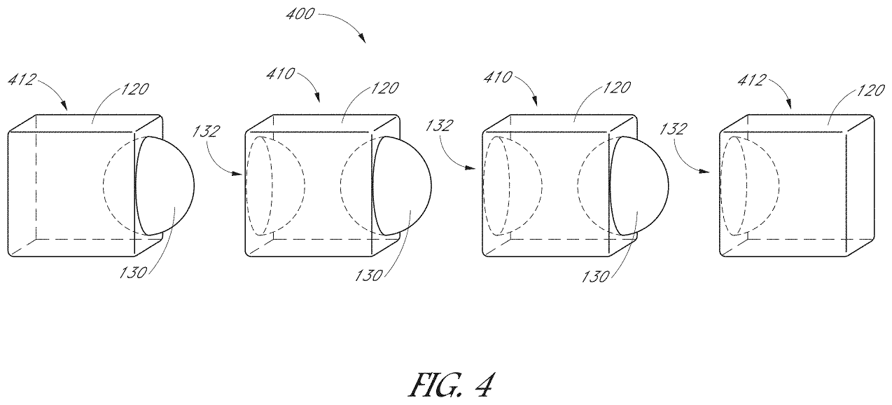

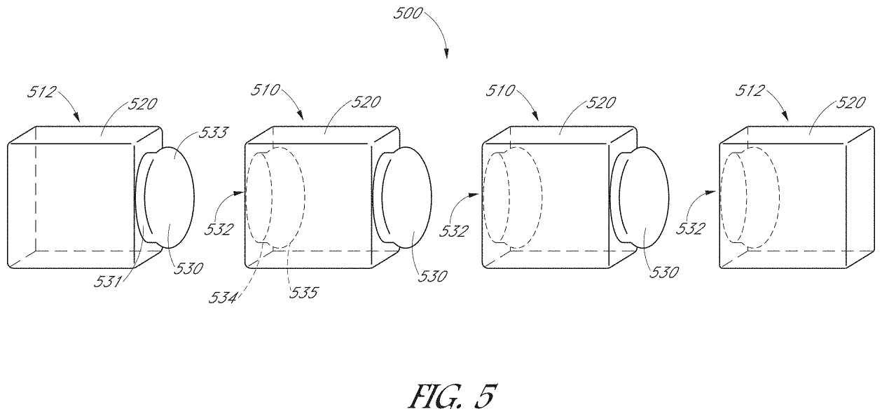

FIGS. 4 and 5 are perspective views of other embodiments of a modular mandibular prosthetic 400 and 500, respectively shown in disconnected configurations. The prosthetics 400 and 500 may have the same or similar features as any other prosthetic described herein, such as the prosthetics 100, 200, 300, 301, 307, and/or 309, and vice versa.

As shown in FIG. 4, in certain embodiments, the prosthetic 400 includes inner links 410 and end links 412 having the bodies 120. In certain embodiments, the inner links 410 and the right-most end link 412 (as oriented) include the receiving portions 132 formed as spherical concave features. In certain embodiments, the inner links 410 and the left-most end link 412 (as oriented) include the engagement structure 130 formed as a spherical convex feature.

As shown in FIG. 5, in certain embodiments, the prosthetic 500 includes inner links 510 and end links 512 having bodies 520. In certain embodiments, the inner links 510 and the left-most end link 512 (as oriented) include engagement structures 530 formed as mushroom shaped engagement structures. For example, the engagement structures 530 include a relatively narrowed neck region 531 extending outwardly away from an end of a corresponding body 520. A wider end portion 533 is located on the end of the neck region 531 that is farthest from the body 520. The end portion 533 may have a partially spherical volume forming an outer rounded surface.

In certain embodiments, the inner links 510 and the right-most end link 512 (as oriented) include receiving portions 532 corresponding to the engagement structures 530. In certain embodiments, the receiving portions 532 include a narrow opening 534 extending inward into the body 520 to a wider opening region 535. In certain embodiments, the narrow opening 534 corresponds in size and shape to the narrowed neck region 531 of the engagement structure 530. The wider opening region 535 corresponds in size and shape to the wider end portion 533 of the engagement structure 530.

The engagement structure 530 and/or the receiving portion 532 may be flexible to allow the relatively wider end portion 533 of the engagement structure 530 to squeeze through the narrow opening 534 of the receiving portion 532. In certain embodiments, the links 510, 512 may be rotatable relative to each other after the receiving portions 532 have received corresponding engagement structures 530 therein and prior to locking.

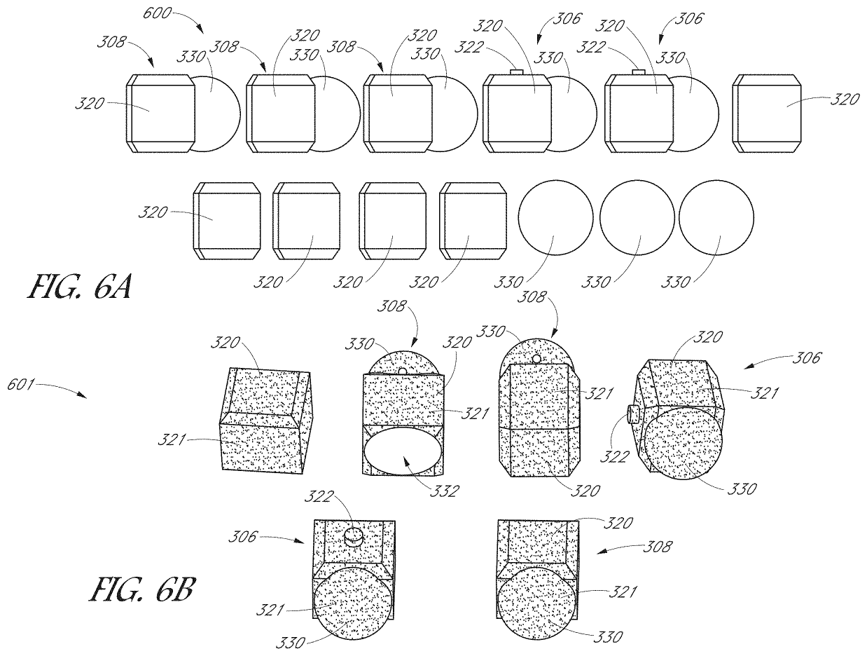

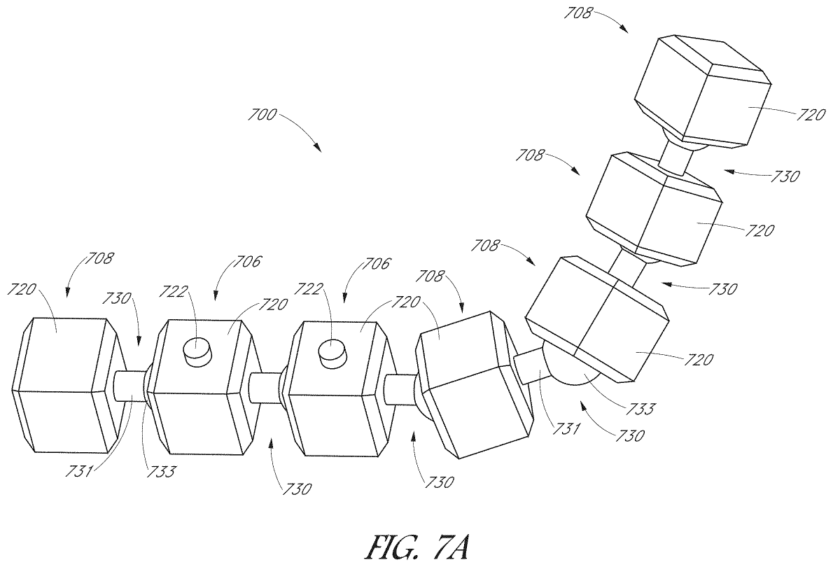

FIGS. 6A and 6B are side and perspective views respectively of other embodiments of modular mandibular prosthetics 600 and 601 respectively, shown prior to connecting the connectable links together. The prosthetics 600 and 601 may have the same or similar features as any other prosthetic described herein, such as the prosthetics 100, 200, 300, 301, 307, 309, 400, and/or 500, and vice versa.

As shown in FIG. 6A, in certain embodiments, the prosthetic 600 includes the links 306, 308 having the bodies 320 and the engagement structures 330. The links 306 include the projections 322. As shown in FIG. 6B, in certain embodiments, the prosthetic 601 includes the links 306, 308 having the bodies 320 and the engagement structures 330. Further, the links 306, 308 include trabeculated tissue surfaces 321 to encourage mandible tissue ingrowth, or as scaffolding for grafting materials to encourage native mandibular growth. The bodies 320 and the engagement structures 330 include the surfaces 321. The surfaces 321 may be scaffolding intended to promote tissue growth for biocompatibility and further securement of the prosthetic in the mandible. The surfaces may also be coarse or smooth to encourage a friction tight locking interface.

FIGS. 7A-7C are perspective views of another embodiment of a modular mandibular prosthetic 700. FIGS. 7A and 7B depict the prosthetic 700 in various connected. configurations. FIG. 7C depicts the prosthetic 700 in an unconnected configuration. The prosthetic 700 may have the same or similar features as any other prosthetic described herein, such as the prosthetics 100, 200, 300, 301, 307, 309, 400. 500, 600, or 601, and vice versa.

In certain embodiments, the prosthetic 700 includes links 706, 708 including bodies 720 with chamfered or rounded edges, and with engagement structures 730 and/or receiving portions 732. The links 706 include bodies 720 having projections 722. In certain embodiments, the engagement structures 730 are swivel-type engagement structures. In certain embodiments, each engagement structure 730 includes a neck 731 extending outwardly form the body 720 to a spherical end portion 733. In certain embodiments, the neck 731 has a narrower width than a maximum width of the spherical end portion 733. The neck 731 may have a circular profile as shown, or other rounded profile. The spherical end portion 733 may include a majority of a complete sphere, for example more than 50% of a sphere. The spherical end portion 733 is received into the receiving portion 732 of an adjacent link 706 or 708 and is secured therein.

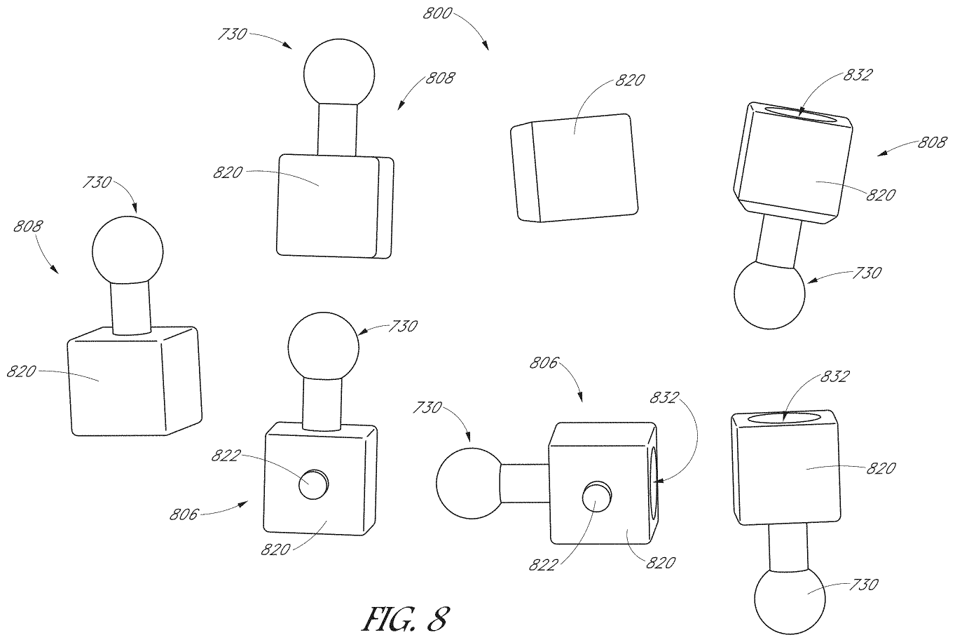

FIG. 8 is a perspective view of another embodiment of a modular mandible prosthetic 800. The prosthetic 800 may have the same or similar features as any other prosthetic described herein, such as the prosthetics 100, 200, 300, 301, 307, 309, 400, 500, 600, 601, or 700, and vice versa. In certain embodiments, the prosthetic 800 includes the swivel engagement structures 730. In certain embodiments, the prosthetic 800 includes links 806 and 808, which are shown disconnected from each other. The links 806, 808 include cuboidal bodies 820 but are not limited to the illustrated shape.

Some or all of the bodies 820 may include a receiving portion 832 and/or the engagement structures 730 extending outwardly therefrom. In certain embodiments, the links 806 include projections 822. In certain embodiments, the bodies 820 are cuboidal, with one of the bodies 820 having chamfered edges, and the remaining bodies not having chamfered edges. More than one, or none, of the bodies may have the chamfered edges.

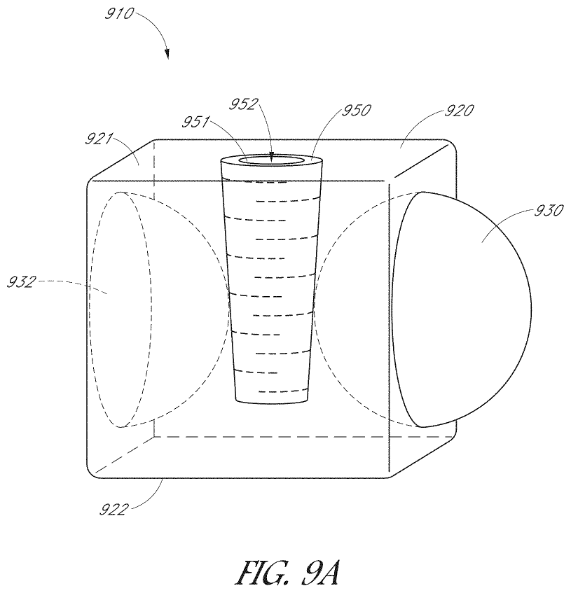

FIGS. 9A and 9B are perspective views of an embodiment of a connecting link 910. The link 910 may have the same or similar features as any other links described herein, such as the links 110, 112, 306, 308, 410, 412, 510, 512, 706, 708, 806 or 808, and vice versa. The link 910 includes a body 920 having a receiving portion 932 and an engagement structure 930.

In certain embodiments, the body 920 further includes a receptacle 950. In certain embodiments, the receptacle 950 extends downward from a first surface 921, such as an upper surface, of the body 920 into the body 920. In certain embodiments, the receptacle 950 has a frustoconical shape that tapers to a smaller width into the body 920. The receptacle 950 may be cylindrical, a segmented polygonal shape, other suitable shapes, or combinations thereof. The receptacle 950 may have an outer rounded wall as shown. In certain embodiments, the body 920 can have the implant already manufactured into the design. In such an embodiment, the crown is then screwed onto the implant.

The receptacle 950, for example the outer wall, defines a channel 952. The channel 952 may be frustoconical as shown, or have the other shapes described above with respect to the receptacle 950. In certain embodiments, the channel 952 extends from an opening 951 at an entrance to the channel 952 located at the upper surface 921 of the body 920. The channel 952 extends into the body 920. In certain embodiments, the channel 952 terminates above the opposite second surface 922, such as a lower surface, of the body 920. The channel 952 may extend farther or less far than shown, for example for 25%, 50%, 75%, or for other percentages, of the depth of the body 920 in the direction from the first surface 921 to the second surface 922.

As shown in FIG. 9B, the channel 952 can be configured to receive an implant 954. The implant 954 may be a tooth, such as a prosthetic tooth. In certain embodiments, the implant 954 may be secured with the receptacle 950 via friction fitting, bonding, fastening, screwing, using other suitable mechanical attachment methods, or combinations thereof.

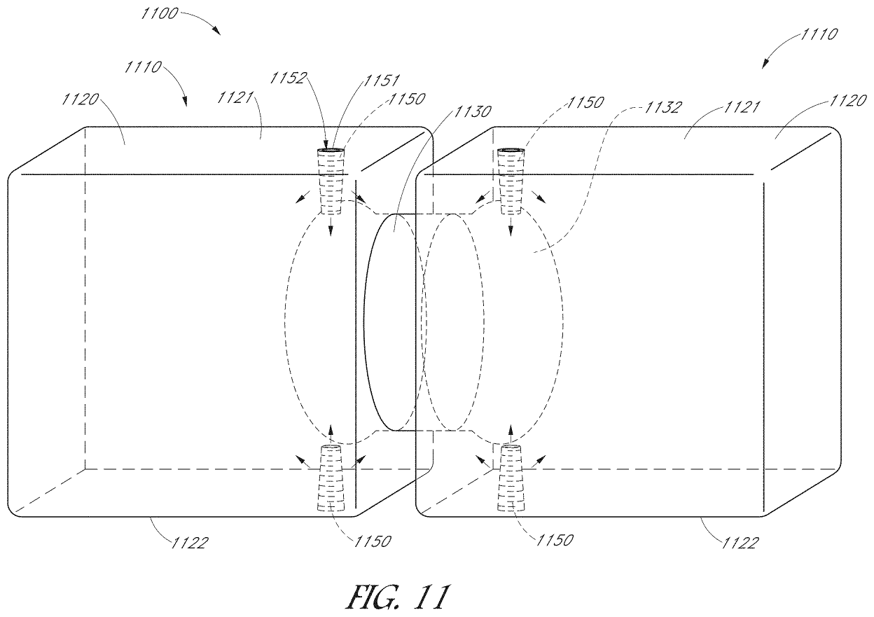

In some embodiments, one or more receptacles 950 may be included and configured to receive an expansion or compression member therein, as described in further detail herein, for example with respect to FIGS. 11-12B.

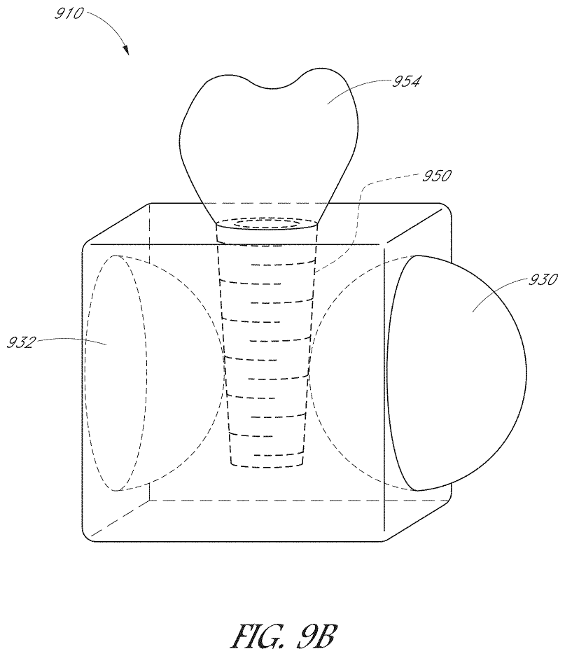

FIG. 10 is a perspective view of another embodiment of a connecting link 1010. The link 1010 may have the same or similar features as any other links described herein, such as the links 110, 112, 306, 308, 410, 412, 510, 512, 706, 708, 806, 808 or 910, and vice versa.

The link 1010 includes a body 1020 having an engagement structure 1030. In certain embodiments, the engagement structure 1030 may be received into a receiving portion 1032 of the body 1020. In certain embodiments, the engagement structure 1030 includes a plurality of targets 1035. There are five targets 1035. There may be one, two, three, four, six, seven, eight, nine, ten or more of the targets 1035. The targets 1035 may be regions of the engagement structure 1030 configured to each receive an expansion or compression member therein to cause the prosthetic, in which the link 1010 is used, to take a locked state. The targets 1035 may include openings, such as holes, channels, shallow grooves, or the like, into which expansion or compression members may be received, as further described herein. There may be multiple targets 1035 within the spherical subunit to accommodate the fact that with rotation of the ball and socket interface, the exposed targets 1035 may not always be in the standard location. With multiple targets 1035, there will be more opportunities for the expansion or compression member to be exposed for easy access.

FIG. 11 is a perspective view another embodiment of a modular mandibular prosthetic 1100. In certain embodiments, the prosthetic 1100 includes two connecting links 1110. The prosthetic 1100 may include more than two links 1110. Each link 1110 includes a body 1120 having a first surface 1121, such as an upper surface, and a second surface 1122, such as a lower surface. The first and second surfaces 1121, 1122 may be on any of the outer surfaces or faces of the body 1120.

The left link 1110 (as oriented in the figure) includes an engagement structure 1130. In certain embodiments, the engagement structure 1130 includes a mushroom shaped configuration, which may be similar to the engagement structure 530 as described with respect to FIG. 5. Any of the other engagement structures described herein may be used with the links 1110.

In certain embodiments, the prosthetic 1100 includes a plurality of expansion or compression members 1150. There are four expansion or compression members 1150 as shown. There may be one, two, three, five, six, seven, eight, nine, ten or more expansion or compression members 1150. The expansion or compression members 1150 may include an opening 1151 and define a channel 1152 therethrough, similar to the receptacles 950. In some embodiments, the expansion or compression members 1150 may not include the opening 1151 and/or channel 1152. The expansion or compression members 1150 may include attachment features, such as slots or grooves, at outer ends thereof and that are configured to be mechanically grabbed by a tool, such as a screw driver or drill, for fastening, e.g., rotating, the expansion or compression members 1150 into the body 1120. In certain embodiments, the body 1120 may include openings at the surfaces 1121 and/or 1122 configured to facilitate entry and securement of the expansion or compression members 1150. In certain embodiments, the openings in the bodies 1120 may be pilot holes, or holes with smaller widths than the expansion or compression members 1150.

In certain embodiments, as the expansion or compression members 1150 are secured with the body 1120, the expansion or compression member 1150 may expand. In some embodiments, the portions of the body 1120 surrounding the expansion or compression member 1150 may expand. The body 1120 may be formed of a material that will expand to secure the expansion or compression member 1150 therein without cracking or otherwise failing. In certain embodiments, the expansion or compression member 1150 may have a larger width and/or depth than an opening in the body 1120 into which the expansion or compression member 1150 is secured, such that the adjacent region of the body 1120 expands slightly.

The expansion or compression members 1150 are engaged with the body 1120 and with the engagement structure 1130. The engagement structure 1130 may include various targets or locations, such as the targets 1035 described with respect to FIG. 10, that may receive the expansion or compression member 1150 therein.

As the expansion or compression members 1150 are secured with the engagement structure 1130, the engagement structure 1130 expands. The portion of the engagement structure 1130 surrounding the expansion or compression member 1150 may expand. The engagement structure 1130 may be formed of a material that will expand to secure the expansion or compression member 1150 therein without cracking or otherwise failing. The expansion or compression member 1150 may have a larger width and/or depth than an opening in the engagement structure 1130 into which the expansion or compression member 1150 is secured, such that the adjacent region of the engagement structure 1130 expands slightly.

In certain embodiments, the expansion or compression member 1150 may fixedly attach to the narrow neck region as well as the wider end portion of the mushroom shaped engagement structure 1130. In certain embodiments, the expansion or compression member 1150 may engage with a spherical portion, a bulbous portion, a cylindrical portion, or any other portion, of the various engagement structures described herein.

In certain embodiments, the expansion or compression member 1150 thus fixedly attaches to both the body 112.0 and to the engagement structure 1130. In this manner the orientation of the engagement structure 1150 is fixed relative to the body 1120. Thus the prosthetic links 1110 can be oriented in a desired configuration by the surgeon and then the expansion or compression members 1150 secured with the prosthetic 1100 to cause the prosthetic 1100 to take a rigid, locked state. In the locked state, the links 1110 of the prosthetic 1100 are not freely movable relative to each other. In certain embodiments, “freely movable” means that at least more than the force due to gravity acting on the links 1110 is required to move one link 1110 relative to the adjacent link 1110. In certain other embodiments, “freely movable” means that the prosthetic 1100 can also withstand the forces of mastication acting on the links 1110 before one link 1110 moves relative to the adjacent link 1110.

A level of resistance to moving the first connector link 1110 relative to the second connector link 1110 increases when the prosthetic 1100 is moved from the unlocked state to the locked state. The level of resistance to movement may therefore be increased after engaging the expansion or compression members 1150 with the prosthetic 1100. In certain embodiments, the expansion or compression members 1150 may be engaged with the body 1120 and the engagement structure 1130 to increase the level of resistance.

FIGS. 12A and 12B are sequential side views of a spherical engagement structure 1230 shown respectively before and after tightening of the expansion or compression member 1150 into the engagement structure 1230. The engagement structure 1230 may include any of the features of the various engagement structures described herein, such as the engagement structure 130, 330, 530, 730. 930, 1030 or 1130, and vice versa.

As shown in FIG. 12A, prior to engaging the expansion or compression member 1150 with the engagement structure 1230, a gap 1232 exists between an outer surface 1153 of the expansion or compression member 1150 and an opening 1231 of the engagement structure 1230. As shown in FIG. 12B, after engaging the expansion or compression member 1150 with the engagement structure 1230, for example by rotating as shown, the gap 1232 is entirely or partially gone and the engagement structure 1230 expands in microincrements. The engagement structure 1230 may expand in the region surrounding the expansion or compression member 1150. The engagement structure 1230 may expand in or more directions by 0.001″ (inches) or more, by 0.005″ or more, by 0.010″ or more, by 0.50″ or more, by 0.01 inches or more, or by greater amounts.

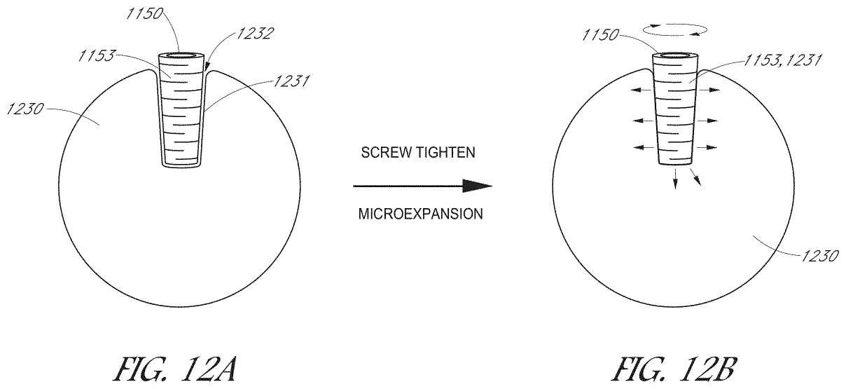

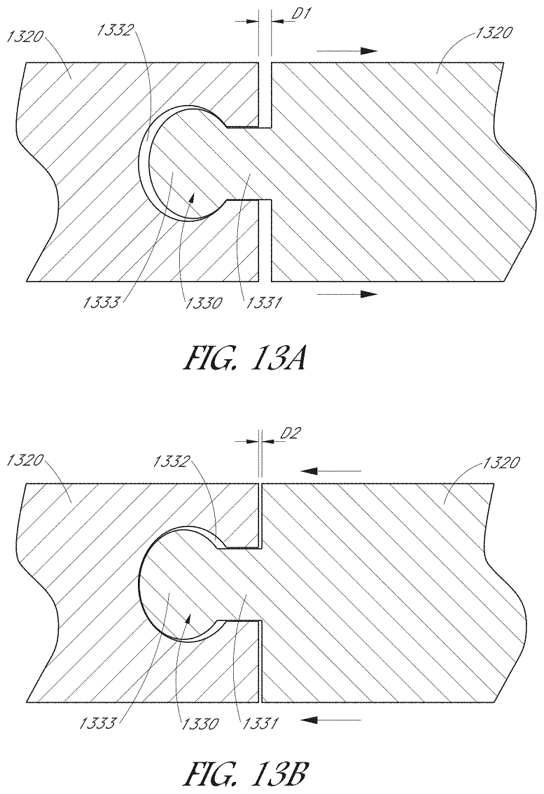

FIGS. 13A and 13B are cross-section views of other embodiments of two connecting links 1320 having an engagement structure 1330 and corresponding receiving portion 1332. The engagement structure 1330 includes a narrow neck 1331 and a wider end portion 1333. The engagement structure 1330 and receiving portion 1332 may be similar respectively to the engagement structure 730 and receiving portion 732 or 832 described herein with respect to FIGS. 7A-8. The receiving portion 1332 includes an opening with a longer axial length than the corresponding axial length of the end portion 1333 of the engagement structure 1330. The width of the receiving portion 1332 may be similarly over-sized. There is thus axial movement that allows for fine adjustments.

In FIGS. 13A and 13B, the links 1320 are shown respectively in first and second axial positions. In the first axial position, the bodies 1320 are separated by a first distance In the second axial position, the bodies 1320 are separated by a second distance D2. The first distance Di is greater than the second distance D2. The axial movement between the receiving portion 1332 and the engagement structure 1330 allows for this fine adjustment. The distances D1 and/or D2 may be 0.1″ (inches), 0.15″, 0.2″, 0.25″, 0.3″, 0.35″, 0.4″, 0.45″, 0.5″, about any of these values, between any of these values, or less than or greater than any of these values. The engagement structure 1330 may be configured to receive one or more expansion or compression members in the first or second axial position, or in any position therebetween. In certain embodiments, the engagement structure 1330 may receive the expansion or compression member 1150 as described with respect to FIGS. 11-12B.

FIGS. 14A and 14B are cross-section views of other embodiments of two connecting links 1420 having an engagement structure 1430 and corresponding receiving portion 1432. In certain embodiments, the engagement structure 1430 includes a narrow neck 1431 and a wider end portion 1433. The engagement structure 1430 and receiving portion 1432 may be similar respectively to the engagement structure 730 and receiving portion 732 or 832 described herein with respect to FIGS. 7A-8.

In certain embodiments, the receiving portion 1432 includes an opening with a longer axial length than the corresponding axial length of the end portion 1433 of the engagement structure 1430. The width of the receiving portion 1432 maybe similarly over-sized. In certain embodiments, the receiving portion 1432 includes an inner region 1432A and an outer region 1432B. The inner region 1432A is located axially inwardly of the outer region 1432B. In certain embodiments, each region 1432A, 1432B includes a rounded, e.g. spherical or ovular, opening therein configured to receive and secure the end portion 1433 of the engagement structure 1430 therein. There is thus axial movement that allows for coarse adjustments.

In FIGS. 14A and 14B, the links 1420 are shown respectively in first and second axial positions. In the first axial position, the end portion 1433 is located within the outer region 1432B and the bodies 1320 are separated by a first distance D1. In the second axial position, the end portion 1433 is located within the inner region 1432A and the bodies 1420 are separated by a second distance D2. The first distance D1 is greater than the second distance D2. The axial distance between the regions 1432A, 1432B and the engagement structure 1430 allows for this coarse or stepped adjustment. The distances D1 and D2 may be 0.1″ (inches), 0.25″, 0.5″, 0.75″, about any of these values, between any of these values, or less than or greater than any of these values.

In certain embodiments, the engagement structure 1430 may be configured to receive one or more expansion or compression members in the first or second axial position, or in any position therebetween. The engagement structure 1430 may receive the expansion or compression member 1150 as described with respect to FIGS. 11-12B. In some embodiments, the regions 1432A, 143213 may include the axial movement described with respect to the receiving portion 1332 (FIGS. 13A-B), such that both coarse or stepped adjustments as well as fine adjustments may be made with the prosthetic.

The links 1320 and 1420 may have the same or similar features as any other links described herein, such as the links 110, 112, 306, 308, 410, 412, 510, 512, 706, 708, 806, 808 or 910, and vice versa.

FIGS. 15A and 15B are various views respectively of embodiments of a connecting link body 1520 and prosthetic 1550 having various trabeculated tissue surfaces 1521 to encourage mandible tissue ingrowth or to act as a scaffolding for grafting and tissue engineered materials. FIG. 15A depicts the body 1520 having the tissue surface 1521 on all six sides of the cuboidal body 1520 (only some of the sides are visible). The tissue surface 1521 may be similar to the surface 321 shown and described with respect to FIG. 6B.

FIG. 15B depicts the prosthetic 1500 including two of the bodies 1520 connected via an engagement structure 1530. The bodies 1521 and engagement structure 1530 are covered with the tissue or graft surface 1521. After a period of time, native tissue 1522 has grown onto and around the tissue surfaces 1521 to cover the prosthetic 1500.

FIG. 16 is a perspective view of another embodiment of a body 1620. The body 1620 may include any of the features of the other bodies describe herein, such as the body 120, 320, 520, 720, 820, 920, 1020, 1120, 1320, 1420 or 1520, and vice versa. The body 1620 includes a hollow structure. A plurality of beams 1622 (only some of which are labeled for clarity) form the outer structure of the body 1620. A hollow interior 1625 is defined by the body 1620. In certain embodiments, the beams 1622 surround and form the interior 1625. The beams 1622 may include features to attach to or with any of the engagement structures described herein. The beams 1622 may form or support any of the receiving portions described herein. The beams 1622 may provide a scaffolding for graft materials, provide a light-weight alternative, and enhance strength.

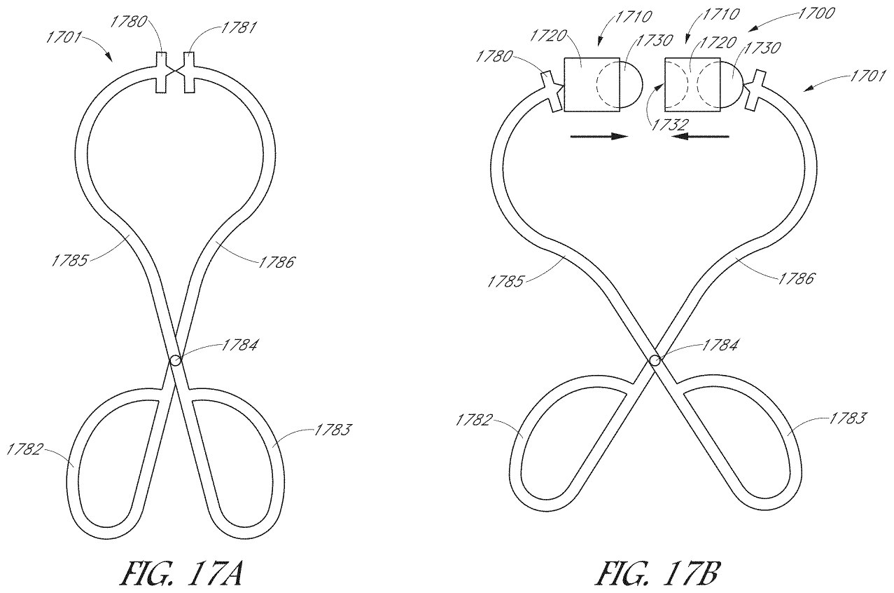

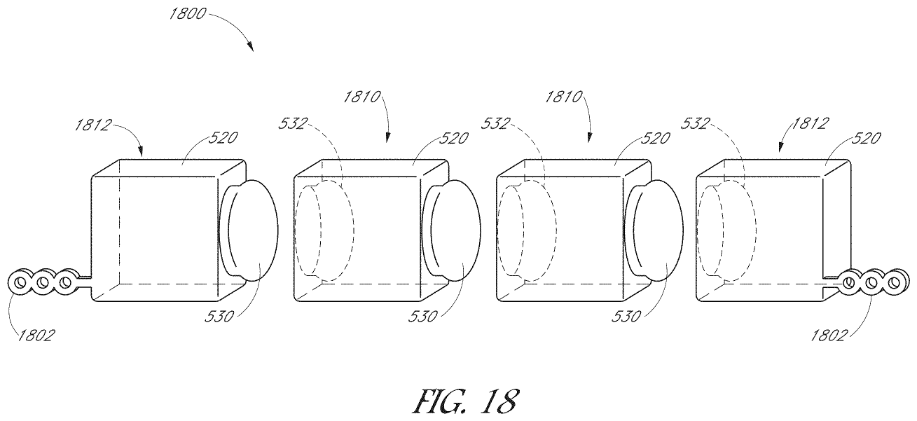

FIGS. 17A and 17B are sequential side views of an embodiment of a tool 1701 that may be used to connect the links 1710 of a modular mandibular prosthetic 1700 together. The prosthetic 1700 includes the links 1710 having a body 1720 with an engagement structure 1730 and/or a receiving portion 1732. The tool 1701 includes gripping portions 1780 and 1781. Handle portions 1782 and 1783 may be held by a human hand and moved closer together to cause corresponding members 1785, 1786 to be rotated about a hinge 1784. The rotation opens and closes the tool 1701 causing the gripping portions 1780, 1781 to move farther apart and closer together.

As shown in FIG. 17B, the gripping portion 1780 may grasp or otherwise contact the body 1720 of one of the links 1710. The gripping portion 1781 may grasp or otherwise contact the engagement structure of the other of the links 1710. The handle portions 1782, 1783 may then be moved closer together to cause the members 1785, 1786 to rotate about the hinge 1784 and thereby bring the gripping portions 1780, 1781 closer together. The gripping portions 1780, 1781 then cause the links 1.710 to be brought closer together and a force is further applied to the handle portions 1782, 1783 to cause a compressive force to be applied by the gripping portions 1780, 1781 to the links 1710. The compressive force causes the engagement structure 1730 to engage with the receiving portion 1732.

FIG. 18 is a perspective view of another embodiment of a modular mandibular prosthetic 1800. The prosthetic 1800 may have the same or similar features as any other prosthetic described herein, such as the prosthetics 100, 200, 300, 301, 307, 309, 400, 500, 600, 601, 700, 800, 1100 or 1800, and vice versa. The prosthetic 1800 includes inner links 1810 and end links 1812. In certain embodiments, the links 1810, 1812 include the body 520 with the engagement structure 530 and/or the receiving portion 532. In certain embodiments, the end links 1812 can include other embodiments of the attachments 1802. In the illustrated embodiment, the attachments 1802 are integral features of the bodies 520. In certain embodiments, the attachments 1802 may be formed together with the corresponding body 520, for example machined from the same piece of material or additively manufactured together. The attachment 1802 may be formed separately and then integral connected with the body 520, for example by adhesive, material melting and solidification, or other suitable techniques.

While the above detailed description has shown, described, and pointed out novel features of the invention as applied to various embodiments, it will be understood that various omissions, substitutions, and changes in the form and details of the device or process illustrated may be made by those skilled in the art without departing from the spirit of the invention. As will be recognized, the present invention may be embodied within a form that does not provide all of the features and benefits set forth herein, as some features may be used or practiced separately from others. The scope of the invention is indicated by the appended claims rather than by the foregoing description. All changes which come within the meaning and range of equivalency of the claims are to be embraced within their scope.

The methods described herein are illustrative only. A person of skill in the art will understand that the steps, decisions, and processes embodied in the methods and any corresponding flowcharts described herein may be performed in an order other than that described herein. Thus, the particular flowcharts and descriptions are not intended to limit the associated processes to being performed in the specific order described.

It will also be appreciated by those of skill in the art that parts included in one embodiment are interchangeable with other embodiments; one or more parts from a depicted embodiment may be included with other depicted embodiments in any combination. For example, any of the various components described herein and/or depicted in the Figures may be combined, interchanged or excluded from other embodiments.

With respect to the use of substantially any plural and/or singular terms herein, those having skill in the art may translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches. For example, terms such as about, approximately, substantially, and the like may represent a percentage relative deviation, in various embodiments, of ±1%, ±5%, ±10%, or ±20%.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives corning within the true scope and spirit of the invention as embodied in the attached claims.

What is claimed is:

1. A modular mandibular prosthetic for replacing at least a portion of a patient's mandible, comprising:
at least a first connector link and a second connector link, both of the first and second connector links forming at least a portion of the modular mandibular prosthetic, the modular mandibular prosthetic having an unlocked state and a locked state, each of the first and second connector links having a body, a first end and a second end, the first end forming a first engagement structure and the second end forming a second engagement structure, the first engagement structure of the first connector link being sized and shaped to be received by the second engagement structure of the second connector link; and
an expansion or compression member configured to engage with at least one of the first connector link and the second connector link when the first engagement structure of the first connector link is received by the second engagement structure of the second connector link to move the modular mandibular prosthetic from the unlocked state to the locked state.

2. The modular mandibular prosthetic of claim 1, wherein a level of resistance to moving the first connector link relative to the second connector link increases when the modular mandibular prosthetic is moved from the unlocked state to the locked state.

3. The modular mandibular prosthetic of claim 1, wherein the body of each of the first connector link and the second connector link has a general cuboidal shape.

4. The modular mandibular prosthetic of claim 1, wherein the body of each of the first connector link and the second connector link has a unitary structure.

5. The modular mandibular prosthetic of claim 1, wherein the body of each of the first connector link and the second connector link has a monolithic structure.

6. The modular mandibular prosthetic of claim 1, wherein the first engagement structure of the first connector link has a convex spherical shape and the second engagement structure of the second connector link has a concave spherical shape.

7. The modular mandibular prosthetic of claim 1, wherein the first engagement structure of the first connector link has a ball shape and the second engagement structure of the second connector link has a socket shape.

8. The modular mandibular prosthetic of claim 1, wherein the body of the first connector link has a receptacle disposed between the first end and the second end, the receptacle being sized and shaped to receive a dental implant.

9. The modular mandibular prosthetic of claim 8, wherein the receptacle is disposed between the first engagement structure and the second engagement structure.

10. The modular mandibular prosthetic of claim 1, further comprising at least one end attachment, the at least one end attachment being configured to connect an end of the portion of the modular mandibular prosthetic to a portion of the patient's mandible.

11. The modular mandibular prosthetic of claim 10, wherein the at least one end attachment has a generally planar shape.

12. The modular mandibular prosthetic of claim 1, wherein the second engagement structure of the first connector link is sized and shaped to receive the first engagement structure of the second connector link.

13. The modular mandibular prosthetic of claim 1, wherein the expansion or compression member is a screw.

14. The modular mandibular prosthetic of claim 1, wherein the expansion or compression member engages with the first connector link by rotating the expansion or compression member relative to the first connector link.

15. The modular mandibular prosthetic of claim 1, wherein the expansion or compression member engages with the body of the first connector link by penetrating the body.

16. The modular mandibular prosthetic of claim 1, wherein the expansion or compression member penetrates the first engagement member of the first connector link at a location.

17. The modular mandibular prosthetic of claim 16, wherein the location is between the body of the first connector link and the body of the second connector link.

18. The modular mandibular prosthetic of claim 1, wherein the expansion or compression member engages with the second connector link by rotating the expansion or compression member relative to the second connector link.

19. The modular mandibular prosthetic of claim 1, wherein the first connector link is fixed with respect to the second connector link when the portion of the modular mandibular prosthetic is in the locked state.

20. The modular mandibular prosthetic of claim 1, wherein the second engagement structure of the second connector link is sized and shaped to receive the first engagement structure of the first connector link in at least a first position and a second position so that an overall length of the first connector link and the second connector link when the modular mandibular prosthetic is in the first position is greater than an overall length of the first connector link and the second connector link when the modular mandibular prosthetic is in the second position.

21. The modular mandibular prosthetic of claim 20, wherein moving between the first position and the second position is achieved by rotating the first connector link relative to the second connector link.

22. The modular mandibular prosthetic of claim 20, wherein moving between the first position and the second position is achieved by axially moving the first connector link relative to the second connector link.

23. The modular mandibular prosthetic of claim 1, wherein the expansion or compression member is releasable.

24. The modular mandibular prosthetic of claim 1, wherein the portion of the modular mandibular prosthetic maintains an assembled state when the modular mandibular prosthetic is in the unlocked state and the expansion or compression member is disengaged from both of the first connector link and the second connector link.

25. A modular mandibular prosthetic for replacing at least a portion of a patient's mandible, the patient's mandible having a lingual surface and a facial surface, comprising:
at least a first connector link and a second connector link, both of the first and second connector links forming at least a portion of the modular mandibular prosthetic, the modular mandibular prosthetic having an unlocked state and a locked state, each of the first and second connector links having a body, a first end and a second end, the first end forming a first engagement structure and the second end forming a second engagement structure, the first engagement structure of the first connector link being configured to be received by the second engagement structure of the second connector link, the first engagement structure and the second engagement structure lying in a plane between the lingual surface and the facial surface; and
an expansion or compression member configured to engage with at least one of the first connector link and the second connector link when the first engagement structure of the first connector link is received by the second engagement structure of the second connector link.

26. A modular mandibular prosthetic for replacing at least a portion of a patient's mandible, the modular mandibular prosthetic having an unlocked state and a locked state, comprising:
a first connector link having a body, a first end and a second end, the first end forming a first engagement structure and the second end forming a second engagement structure, the first connector link forming at least a first portion of the modular mandibular prosthetic; and
a second connector link having a body, a first end and a second end, the first end forming a first engagement structure and the second end forming a second engagement structure, the second connector link forming at least a second portion of the modular mandibular prosthetic,
the first engagement structure of the first connector link being sized and shaped to be received by the second engagement structure of the second connector link when the modular mandibular prosthetic is in both the unlocked state and the locked state.

27. The modular mandibular prosthetic of claim 26, wherein the first connector link is movable with respect to the second connector link when the modular mandibular prosthetic is in the unlocked state.

28. The modular mandibular prosthetic of claim 27, wherein the movement of the first connector link is a swivel.

29. The modular mandibular prosthetic of claim wherein the swivel is in three dimensions.

30. The modular mandibular prosthetic of claim 26, further comprising an expansion or compression member configured to engage with at least one of the first connector link and the second connector link when the first engagement structure of the first connector link is received by the second engagement structure of the second connector link to move the modular mandibular prosthetic from the unlocked state to the locked state.
